(19) United States
(12) Patent Application Publication
(10) Pub. No.: US 2022/0299957 A1
(43) Pub. Date: Sep. 22, 2022

(54) CONSTRUCTING A 3D PHANTOM WITH LIQUID HYDROGEL

(71) Applicant: Novocure GmbH, Root D4 (CH)

(72) Inventors: Yoram WASSERMAN, Haifa (IL); Stas OBUCHOVSKY, Haifa (IL); Nataliya KUPLENNIK, Haifa (IL)

(21) Appl. No.: 17/698,777

(22) Filed: Mar. 18, 2022

Related U.S. Application Data
(60) Provisional application No. 63/162,921, filed on Mar. 18, 2021.

(51) Int. Cl. G05B 17/02; G16C 20/30; G09B 23/18

(52) U.S. Cl. CPC G05B 17/02; G16C 20/30; G09B 23/182; A61N 1/0496

(57) ABSTRACT

A hydrogel phantom is herein described. The hydrogel phantom includes a plurality of adjacently disposed hydrogel elements. A first one of the hydrogel elements has a first electrical impedance and a second one of the hydrogel elements has a second impedance. The first impedance is different from the second impedance.

### CROSS REFERENCE TO RELATED APPLICATIONS/INCORPORATION BY REFERENCE STATEMENT

The present patent application claims priority to the provisional patent application identified by U.S. Ser. No. 63/162,921, filed on Mar. 18, 2021. The entire content of U.S. Ser. No. 63/162,921 is hereby incorporated herein by reference.

### STATEMENT REGARDING FEDERALLY SPONSORED RESEARCH OR DEVELOPMENT

Not Applicable.

### BACKGROUND

Tumor Treating Fields (TTFields or TTFs) are low intensity (e.g., 1-3 V/cm) alternating electric fields within the intermediate frequency range (100-500 kHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields are typically delivered through two pairs of transducer arrays that generate perpendicular fields within the treated tumor; the transducer arrays that make up each of these pairs are positioned on opposite sides of the body part that is being treated. TTFields are approved for the treatment of glioblastoma multiforme (GBM), and may be delivered, for example, via the OPTUNE® system (Novocure Limited, St. Helier, Jersey), which includes transducer arrays placed on the patient's shaved head.

Each transducer array used for the delivery of TTFields in the OPTUNE® device comprises a set of non-conductive ceramic disk electrodes, which are coupled to the patient's skin (such as, but not limited to, the patient's shaved head for treatment of GBM) through a layer of conductive medical gel. To form the ceramic disk electrodes, a conductive layer is formed on a top surface of nonconductive ceramic material. A bottom surface of the nonconductive ceramic material is coupled to the conductive medical gel.

One approach to applying the TTField in different directions is to apply the field between a first set of electrodes for a period of time, then applying a field between a second set of electrodes for a period of time, then repeating that cycle for an extended duration (e.g., over a period of days or weeks). In order to generate the TTFields, current is applied to each electrode of the transducer array. The TTFields interact with the patient and one of more of the patient's organs based on the electrical conductivity of each of the patient's organs. As the TTField interacts with the patient, the field may change shape based in part on the electrical conductivity and relative position of each of the patient's organs. Because the electrical conductivity of each organ of a patient modifies the TTField shape and a particular TTField shape may be needed to effectively target a tumor, it is important to be able to determine how the applied TTField is shaped within a patient.

To date, there has not been a way to measure actual TTField shape in a patient, without computer simulations; however, computer simulations or other models are reliant on programming techniques and estimations, and cannot show the actual TTField shape expected in a patient.

Because the electrical conductivity of each organ of a patient modifies the TTField shape and a particular TTField shape may be needed to effectively target a tumor, new and improved assemblies and methods of using a physical 3D model to determine real-world interactions between the TTField and various organs are desired. It is to such assemblies and methods of producing and using the same, that the present disclosure is directed.

### BRIEF DESCRIPTION OF THE DRAWINGS

FIG. 1 is an exemplary embodiment of a schematic diagram of electrodes as applied to living tissue.

FIG. 2 is an exemplary embodiment of an electronic device configured to generate a TTField.

FIG. 3A is a cross-sectional diagram of an exemplary embodiment of a hydrogel phantom.

FIG. 3B is a cross-sectional diagram of another exemplary embodiment of a hydrogel phantom.

FIG. 4A is a diagram of an exemplary embodiment of a gel application system constructed in accordance with the present disclosure.

FIG. 4B is a diagram of an exemplary embodiment of a second gel application system constructed in accordance with the present disclosure.

FIG. 5 is a process flow diagram of an exemplary embodiment of a hydrogel phantom creation process.

FIG. 6 is a process flow diagram of an exemplary embodiment of a field-generating pad location placement process.

FIG. 7 is a flow chart of an exemplary method for validating a computer simulation using a hydrogel phantom in accordance with the present disclosure.

### DETAILED DESCRIPTION

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth.

As used herein, the term “substantially” means that the subsequently described event or circumstance completely occurs or that the subsequently described event or circumstance occurs to a great extent or degree.

As used herein, the phrases “associated with” and “coupled to” include both direct association/binding of two moieties to one another as well as indirect association/binding of two moieties to one another.

The term “patient” as used herein includes human and veterinary subjects. “Mammal” for purposes of treatment refers to any animal classified as a mammal, including (but not limited to) humans, domestic and farm animals, nonhuman primates, and any other animal that has mammary tissue.

Circuitry, as used herein, may be analog and/or digital components, or one or more suitably programmed processors (e.g., microprocessors) and associated hardware and software, or hardwired logic. Also, “components” may perform one or more functions. The term “component,” may include hardware, such as a processor (e.g., microprocessor), an application specific integrated circuit (ASIC), a field programmable gate array (FPGA), a combination of hardware and software, and/or the like. The term “processor” as used herein means a single processor or multiple processors working independently or together to collectively perform a task.

The term “resistance” or “resistivity” refers to a degree to which a substance or device opposes the passage of electric current causing energy dissipation.

The term “impedance” refers to an effective resistance of an electric circuit or component to alternating current, arising from the combined effects of ohmic resistance and reactance.

The term “conductivity” refers to a degree to which a specified material conducts electricity, calculated as the ration of the current density in the material to the electric field that causes the flow of current. The “conductivity” of a material is the reciprocal of the material's resistivity.

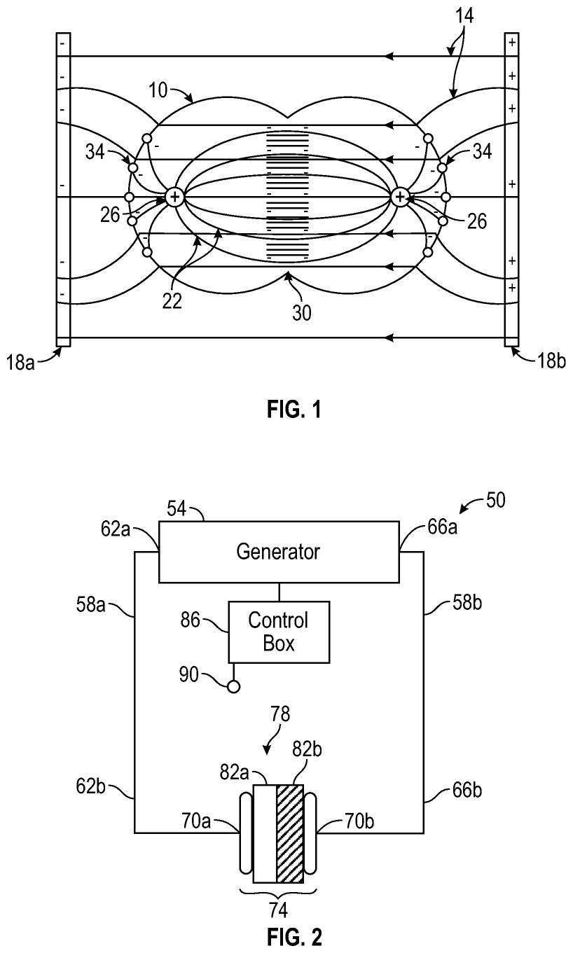

Referring now to the drawings and in particular to FIG. 1, shown therein is a diagram of an exemplary embodiment of a dividing cell 10, under the influence of external TTFields (e.g., alternating fields in the frequency range of about 100 KHZ to about 300 KHZ), generally indicated as lines 14, generated by a first electrode 18a having a negative charge and a second electrode 18b having a positive charge. Further shown are microtubules 22 that are known to have a very strong dipole moment. This strong polarization makes the microtubules 22, as well as other polar macromolecules and especially those that have a specific orientation within the cell 10 or its surroundings, susceptible to electric fields. The microtubules 22 positive charges are located at two centrioles 26 while two sets of negative poles are at a center 30 of the dividing cell 10 and point of attachment 34 of the microtubules 22 to the cell membrane. The locations of the charges form sets of double dipoles and therefore are susceptible to electric fields of differing directions. By adjusting a location of the first electrode 18a and the second electrode 18b in relation to the dividing cell 10, the electric field direction can be adjusted, however, interactions between the electric field and one or more cells or organs intermediate each electrode 18 and the dividing cell may cause changes in the electric field, such as a deflection in the electric field.

Turning now to FIG. 2, the TTFields described above that have been found to advantageously destroy tumor cells are generated by an electronic apparatus 50. FIG. 2 is a simple schematic diagram of the electronic apparatus 50 illustrating major components thereof. The electronic apparatus 50 includes a generator 54 and a pair of conductive leads 58, including first conductive lead 58a and second conductive lead 58b. The first conductive lead 58a includes a first end 62a and a second end 62b. The second conductive lead 58b includes a first end 66a and a second end 66b. The first end 62a of the first conductive lead 58a is conductively attached to the generator 54 and the first end 66a of the second conductive lead 58b is conductively attached to the generator 54. The generator 54 generates desirable electric signals (TT signals) in the shape of waveforms or trains of pulses as an output. The second end 62b of the first conductive lead 58a is connected to a first field-generating pad 70a and the second end 66b of the second conductive lead 58b is connected to a second field-generating pad 70b, that is supplied with the electric signals (e.g., wave forms). Each of the first field-generating pad 70a and the second field-generating pad 70b are in contact with, or otherwise associated with, a field target 74. The electric signals generate an electric field (i.e., TTField) that is capacitively coupled into the field target 74, the TTField having a frequency and an amplitude, to be generated between the first field-generating pad 70a and the second field-generating pad 70b in the field target 74. In one embodiment, the field target 74 is a hydrogel phantom 78 generally comprising two or more hydrogel elements 82a-n shown in FIG. 2 as hydrogel element 82a and hydrogel element 82b, described in more detail below.

Each of the first field generating pad 70a and the second field generating pad 70b includes one or more conductive electrode elements that may be capacitively coupled with the field target 74 by a non-conductive layer. Alternative constructions for the first field generating pad 70a and the second field generating pad 70b may also be used, including, for example, transducer arrays using a non-conductive layer formed of a ceramic element that is disc shaped, or is not disc-shaped, and/or non-conductive layer(s) that use non-ceramic dielectric materials positioned over a plurality of flat conductors. Examples of the latter include polymer films disposed over pads on a printed circuit board or over flat pieces of metal. The first field generating pad 70a and the second field generating pad 70b may also include electrode elements that are not capacitively coupled with the field target 74. In this situation, each of the first field generating pad 70a and the second field generating pad 70b may be implemented using a region of a conductive material that is configured for placement against a person's body, with no insulating dielectric layer disposed between the conductive elements and the body. Examples of the conductive material include, but are not limited to, a conductive film, a conductive fabric, and/or a conductive foam. Other alternative constructions for implementing the first field generating pad 70a and the second field generating pad 70b may also be used, as long as they are capable of delivering TTFields to the person's body. Optionally, a layer of hydrogel may be disposed between the first field generating pad 70a and the field target 74; and the second field generating pad 70b and the field target 74 in any of the embodiments described herein.

The generator 54 generates an alternating voltage wave form at frequencies in the range from about 50 KHZ to about 1 MHZ (preferably from about 100 KHZ to about 300 KHZ) (i.e., the TTFields). The required voltages are such that an electric field intensity in tissue within the target region is in the range of about 0.1 V/cm to about 10V/cm. To achieve this field, the potential difference between the two electrodes 18 of the first field-generating pad 70a and the second field-generating pad 70b is determined by the relative impedances of the system components, i.e., a fraction of the electric field on each component is given by that component's impedance divided by a total circuit impedance.

In certain particular (but non-limiting) embodiments, the first field-generating pad 70a and the second field-generating pad 70b generate an alternating electric field within a target region of the field target 74. The alternating electric field may be selected to emulate one or more sources of electromagnetic radiation. For example, in order to simulate a TTField, the alternating electric field may be selected to emulate the TTField (as described below). In other embodiments, for example, when simulating electromagnetic radiation sourced by a cellular telephone, a cellular telephone communication radio signal is generated as the alternating electric field. Simulating electromagnetic radiation sourced by a cellular telephone may be of particular need when measuring the Specific Absorption Rate of a particular cellular telephone.

In certain particular (but non-limiting) embodiments, when the field target 74 is a patient, the target region typically comprises at least a portion of the patient's body, and may be, for example only, a tumor, a particular cell or cluster of cells that are either the same type or different type, a portion of the patient's body that has foreign bodies such as a virus or a bacteria, and/or the like, and the generation of the alternating electric field selectively destroys or inhibits growth of a tumor. The alternating electric field may be generated at any frequency that selectively destroys or inhibits growth of the tumor.

In order to optimize the electric field (i.e., TTField) distribution, the first field-generating pad 70a and the second field-generating pad 70b (pair of field-generating pads 70) may be configured or oriented differently depending upon the application in which the pair of field-generating pads 70a and 70b are to be used. The pair of field-generating pads 70a and 70b, as described herein, are externally applied to the field target 74. When the field target 74 is a patient, the pair of field-generating pads 70 may be applied to the patient's skin, in order to apply the electric current, and electric field (TTField) thereby generating current within the patient's tissue. Generally, the pair of field-generating pads 70 are placed on the patient's skin by a user such that the electric field is generated across patient tissue within a treatment area. TTFields that are applied externally can be of a local type or widely distributed type, for example, the treatment of skin tumors and treatment of lesions close to the skin surface. Similarly, the electric field applied to the field target 74 can be of a local type or a widely distributed type.

Optionally and according to another exemplary embodiment, the electronic apparatus 50 includes a control box 86 and a temperature sensor 90 coupled to the control box 86, which are included to control the amplitude of the electric field.

When the control box 86 is included, the control box 86 controls the output of the generator 54 causing the output to remain constant at a value preset by the user. Alternatively, the control box 86 sets the output of the generator 54. The temperature sensor 90 may be mechanically connected to and/or otherwise associated with the first field-generating pad 70a or the second field-generating pad 70b so as to sense the temperature of the field target 74 at either one or both of the first field-generating pad 70a or the second field-generating pad 70b.

The conductive leads 58 are standard isolated conductors with a flexible metal shield, preferably grounded thereby preventing spread of any electric field generated by the conductive leads 58. The field-generating pads 70a and 70b may have specific shapes and positioning so as to generate the TTField of a desired configuration, direction, and intensity at the target region of the field target 74 and only there so as to focus the electric field.

The specifications of the electronic apparatus 50 as a whole and its individual components are largely influenced by the fact that at the frequency of the TTFields (e.g., 50 KHZ-500 KHZ), living systems behave according to their “Ohmic’, rather than their dielectric properties.

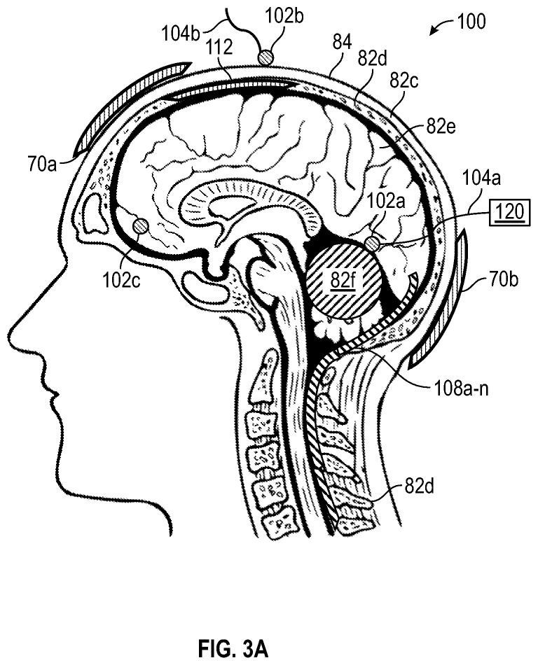

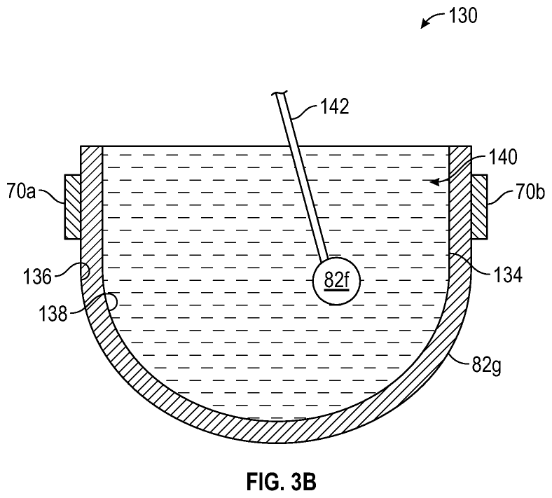

FIGS. 3A and 3B illustrate exemplary embodiments of hydrogel phantoms 78 of FIG. 2. In some embodiments, the hydrogel phantom 78 can be formed in the shape of human or non-human body parts, such as an arm, an elbow, a chest, a leg, a torso, and the like or some combination thereof, or in the form of other types of objects, such as a cell phone, portion of a wall, or the like. In some embodiments, the hydrogel phantom 78 is formed in the shape of a human body, or other animal body. In some embodiments, the hydrogel phantom 78 is formed to be an anatomically-accurate representation of a particular human or other animal or a portion thereof.

Referring to FIG. 3A, shown therein is a cross-sectional diagram of an exemplary embodiment of the hydrogel phantom 78 of FIG. 2 depicted as a hydrogel phantom head 100 (phantom head 100) formed from a plurality of hydrogel elements 82a-n. In the example shown, the hydrogel phantom head 100 is formed from a skin hydrogel element 82c, a bone hydrogel element 82d, and a brain hydrogel element 82e. The phantom head 100 shown in FIG. 3A is depicted as comprising three hydrogel elements 82a-n for simplicity only and may comprise any number of hydrogel elements 82a-n required by the user to appropriately model electrical conductivity of a selected portion of a human body, or a non-human body. Also shown in FIG. 3A is the first field-generating pad 70a and the second field-generating pad 70b on an outer surface 84 of the phantom head 100.

Therefore, in some embodiments, to appropriately model electrical conductivity of a selected biological component, the user should first determine a desired frequency, or range of frequencies, of a signal to be tested so an appropriate conductivity can be selected for the hydrogel phantom 78 to best match the conductivity of the selected biological component. The user may select one or more conductivity values from conductivity of biological components known in the art such as from S Gabriel, in The Dielectric Properties of Biological Tissues: II Measurements in the frequency range of 10 Hz to 20 GHz (S Gabriel et al. 1996 Phys. Med. Biol. 41 2251).

In other embodiments, the user may select a mean conductivity for one or more biological component, such as calculated by Ramon et al. (Ramon C, Gargiulo P, Friðgeirsson EA and Haueisen J (2014) Changes in Scalp Potentials and Spatial Smoothing Effects of Inclusion of Dura Layer in Human Head Models for EEG Simulations. Front. Neuroeng. 7:32. doi:10.3389/fneng.2014.00032). For example only and as calculated in Ramon, the user may select 1.35 E−3 S/cm as a mean conductivity for the skin hydrogel element 82c, 6.25 E−5 S/cm as a mean conductivity for the bone hydrogel element 82d, and 3.334 E−3 S/cm as a mean conductivity for the brain hydrogel element 82e.

In some embodiments, the hydrogel phantom 78 includes one or more support structure (not shown) to provide support for the hydrogel phantom 78. Each of the one or more support structure may be either non-conductive, electrically isolated, or both. In one embodiment, the one or more support structure is selected to cause minimum interference with the generated TTField.

It should be noted that while the exemplary embodiment of the hydrogel phantom 78 of FIG. 2 is depicted as being a hyper-accurate representation of a human head, the hydrogel phantom 78 in some embodiments may be formed of a minimum number of hydrogel elements 82a-n required to model the field target 74 for a desired purpose.

By creating the hydrogel phantom 78, users are able to determine actual values and shape of a TTF field within and/or around the hydrogel phantom 78 resulting from the application of an alternating electric field. For example, by utilizing one or more sensor 102a-n (discussed in more detail below) users are able to determine a magnetic property, such as one or more electric field or electromagnetic field power/intensity; an electrical property such as a voltage, a current, an inductance, a capacitance; a thermal property such as temperature; or a pressure; a force; and/or the like at varying locations within the hydrogel phantom 78. The determined actual values may be recorded for various configurations of the hydrogel phantom 78 and utilized by the user to generate or improve computer simulations. In instances where the hydrogel phantom 78 is representative of a specific patient, the determined actual values may be utilized to improve, or increase the therapeutic benefit of that specific patient's TTField therapy. Moreover, by applying the alternating electric field to the hydrogel phantom 78, users are able to understand how alternating electric fields, such as the TTField, move through a human or non-human body and around various types of tissue and/or bone in an accurate, non-computer-simulation based setting.

In one embodiment, the hydrogel phantom 78 is a polymerized gel (in solid form) that includes two or more hydrogel element 82a-n being polymerized gel having a bulk electron transport agent providing a source of free ions therein to enable electrical conductivity resulting in a volume resistivity. In one embodiment, each hydrogel element 82a-n is formed primarily of a conductive gel or a semi-solid conductive gel.

In the embodiment depicted in FIG. 3A, the phantom head 100 includes three hydrogel elements 82, the skin hydrogel element 82c, the bone hydrogel element 82d, and the brain hydrogel element 82e, with each of the hydrogel elements 82 bonded to at least a portion of another hydrogel element 82. The skin hydrogel element 82c includes a shape, thickness, and volume of human skin, and a substantially uniform electrical resistance/impedance/conductivity mimicking the electrical resistance/impedance/conductivity of human skin. The bone hydrogel element 82d is positioned within the hydrogel phantom 78 in a manner that mimics the location of bone within a human head. The bone hydrogel element 82d includes a shape, thickness, and volume of human bone within a human head, and may include a substantially uniform electrical resistance/impedance/conductivity mimicking the electrical conductivity of human bone. The bone hydrogel element 82d is adjacent to and borders the skin hydrogel element 82c. The brain hydrogel element 82e includes a shape, thickness, and volume of a human brain. The brain hydrogel element 82e may include a substantially uniform electrical resistance/impedance/conductivity mimicking the electrical resistance/impedance/conductivity of the human brain. The brain hydrogel element 82e is partially surrounded by and borders the bone hydrogel element 82d.

Although the hydrogel phantom 78 is described by way of example as having three different types of hydrogel elements, i.e., the skin hydrogel element 82c, the bone hydrogel element 82d, and the brain hydrogel element 82e, the hydrogel phantom 78 can be provided with other types of hydrogel elements, such as a blood vessel hydrogel element, a spinal fluid hydrogel element, a blood hydrogel element, a tumor hydrogel element, or the like. In some embodiments, the hydrogel elements 82 are connected together to form a continuous hydrogel device having regions of varying electrical resistance/conductivity so as to collectively mimic the electrical resistance/impedance/conductivity of a human head.

In one embodiment, each hydrogel element 82a-n is formed primarily of a conductive gel or semi-solid conductive gel such as described below. The plurality of hydrogel elements 82a-n taught herein may be used with modified hydrogels (which includes not only perforations but also recesses, protrusions, etc.) as disclosed in detail in U.S. Patent Application No. 63/020,636 entitled “Conductive Gel Compositions Comprising Bulk Electron Transport Agents and Methods of Production and Use Thereof”, which is hereby incorporated in its entirety.

The conductive gel may be in any form that allows the composition to function in accordance with the present disclosure. For example (but not by way of limitation), each hydrogel element 82a-n may be in the form of a hydrogel or a hydrocolloid.

In one embodiment, each hydrogel element 82a-n may be formed of two or more component hydrogels. Each component hydrogel is a conductive gel having one or more structural, water-soluble polymer, one or more crosslinker, one or more photoinitiators, one or more electrolyte, and one or more additive.

The conductive gel may be formed of any hydrophilic polymer that allows each component hydrogel of each hydrogel element 82a-n to function in accordance with the present disclosure. For example (but not by way of limitation), one or more of the conductive gel may be a polyacrylic acid gel, a povidone gel, or a cellulose gel. In addition, one or more conductive gel may comprise at least one of chitosan, alginate, agarose, methylcellulose, hyaluronan, collagen, laminin, matrigel, fibronectin, vitronectin, poly-1-lysine, proteoglycans, fibrin glue, gels made by decellularization of engineered and/or natural tissues, as well as any combinations thereof. Further, one or more conductive gel may comprise at least one of polyglycolic acid (PGA), polylactic acid (PLA), poly-caprolactone (PCL), polyvinyl alcohol (PVA), polyethylene glycol (PEG), methyl methacrylate, poly(methyl methacrylate) (PMMA), poly(2-hydroxyethyl methacrylate) (PolyHEMA), poly(glycerol sebacate), polyurethanes, poly(isopropylacrylamide), poly(N-isopropylacrylamide), or any combination thereof.

In certain non-limiting embodiments, the conductive gel comprises one or more of the following chemical and structural features/properties: a polymer chain length in a range of from about 1 nm to about 200 nm; a free salt present at a concentration in a range of from about 0.1 mM to about 1 M; a pH in a range of from about 6 pH to about 8 pH; and a volume resistivity of less than about 100 Ohm-in.

The polymer(s) of the conductive gel may be provided with any polymer chain length that allows the conductive gel composition(s) to function as described herein. For example (but not by way of limitation), the polymer chain length may be about in a range of from about 3 nm to about 175 nm, a range of from about 5 nm to about 150 nm, or a range of from about 10 nm to about 125 nm, a range of from about 15 nm to about 100 nm, etc.), and a range that combines two integers that fall between two of the above-referenced values (i.e., a range of from about 3 nm to about 157 nm, etc.).

In some embodiments, the component hydrogel includes one or more electrolyte, such as a purified water electrolyte or free salt.

In one embodiment, each hydrogel element 82a-n may be formed of two or more components. Each component may include one or more structural, water-soluble polymer, one or more crosslinker, one or more photoinitiators, one or more electrolyte, and one or more additive. In one embodiment, the one or more additive of each component may comprise one or more of a humectant, a preservative, and/or the like.

In one embodiment, each component includes one or more crosslinker and one or more photoinitiator. A crosslinker is a structural polymer that provides stabilization of the hydrogel and leads to multidimensional extension of polymeric chains when activated. A photoinitiator is a particular crosslinker that, upon hydrogel curing, activates the one or more crosslinker thereby causing the one or more crosslinker to form into the multidimensional extension of polymeric chains of structural polymers, thus forming a three-dimensional (3D) gel. The crosslinking may be formed by double bonds or functional groups in the structural polymer.

Hydrogel curing is the polymerization of the hydrogel, which may be constructed by the combination of two or more components, generally by applying a curing agent. Before polymerization of the hydrogel, the hydrogel may be a liquid hydrogel. In one embodiment, polymerization can be accomplished by applying UV irradiation of a UV dosage to the hydrogel. The UV dosage may include a duration of irradiation as well as an intensity of irradiation and may be determined based on user requirements, such as a degree of crosslinking, which affects tackiness and conductivity of the hydrogel. That is, by adjusting the UV dosage, e.g., duration of UV irradiation and/or irradiation intensity, the user can adjust cured properties of the hydrogel, such as the conductivity and tackiness of the hydrogel. In another embodiment, polymerization can be accomplished by the application of alternative forms of energy to cure the hydrogel, such as an electron beam or a laser. Applying the electron beam or laser to the hydrogel may result in a high crosslinking speed and efficiency. The duration of the electron beam or laser application as well as intensity of the electron beam or laser application may be modified to adjust cured properties of the hydrogel.

In one embodiment, specific components to form the hydrogel, and/or hydrogel curing is used to provide each of the plurality of hydrogel elements 82a-n with desired cured properties.

For example, in a lab experiment summarized in Table 1 below, a UV curable hydrogel (Product number #JN0917-A) obtained from Polychem UV/EB International Corp. of Taipei Taiwan was used. This UV curable hydrogel includes two components, referred to as a first component and a second component. The first component and the second component were mixed together in three different ratios resulting in a first experimental hydrogel element, a second experimental hydrogel element, and a third experimental hydrogel element. For each experimental hydrogel element, a volume resistivity value was calculated at varying hydrogel curing durations while curing with a UV LED providing light at 365 nm wavelength.

The first experimental hydrogel element was composed of the first component and the second component in a 1:0.3 ratio. At a hydrogel curing duration of 10 min, the volume resistivity value was determined to be 2909 p (Ω per cm); at a hydrogel curing duration of 20 min, the volume resistivity value was determined to be 2909 p (Ω per cm); and, at a hydrogel curing duration of 40 min, the volume resistivity value was determined to be 4160 p (Ω per cm).

The second experimental hydrogel element was composed of the first component and the second component in a 1:0.65 ratio. At a hydrogel curing duration of 10 min, the volume resistivity value was determined to be 272 p (Ω per cm); at a hydrogel curing duration of 20 min, the volume resistivity value was determined to be 356 p (Ω per cm); and, at a hydrogel curing duration of 40 min, the volume resistivity value was determined to be 364 p (Ω per cm). The second experimental hydrogel element is POLYCHEM Advanced UV Curable Conductive #JN0917-A, a fully synthetic polyacrylamide based and chemical cross-linked high-performance hydrogel containing purified water, humectants, and fully synthetic photopolymers. The second experimental hydrogel element had a liquid viscosity of 150±50 cps at 25° Celsius and a pH of between 4.0-7.0.

The third experimental hydrogel element was composed of the first component hydrogel and the second component hydrogel in a 1:0.9 ratio. At a hydrogel curing duration of 10 min, the third experimental hydrogel element still had a mostly liquid form necessitating an additional 5 min of the hydrogel curing duration, at which point the volume resistivity value was determined to be 86 p (Ω per cm); at a hydrogel curing duration of 25 min, the volume resistivity value was determined to be 104 p (Ω per cm); and, at a hydrogel curing duration of 45 min, the volume resistivity value was determined to be 104 p (Ω per cm).

TABLE 1

| | Ratio Used | | |
|---|---|---|---|
| | 1:0.3 | 1:0.65 | 1:0.9 |
| Curing Duration | ρ (Ω per cm) | | |
| 10 min | 2909 | 272 | — |
| 15 min | — | — | 86 |
| 20 min | 2909 | 356 | — |
| 25 min | — | — | 104 |
| 40 min | 4160 | 364 | — |
| 45 min | — | — | 104 |

Referring again to FIG. 3A, each of the skin hydrogel element 82c, the bone hydrogel element 82d, and the brain hydrogel element 82e may be composed of the same or a different ratio of the first component and the second component as another hydrogel element 82. For example, the skin hydrogel element 82c may be composed of a first ratio of the first component and the second component resulting in a first volume resistivity, the bone hydrogel element 82d may be composed of a second ratio of the first component and the second component resulting in a second volume resistivity, and the brain hydrogel element 82e may be composed of a third ratio of the first component and the second component resulting in a third volume resistivity, where the first ratio, the second ratio, and the third ratio may be the same or different and the first volume resistivity, the second volume resistivity, and the third volume resistivity may be the same or different. In one embodiment, one or more of the skin hydrogel element 82c, the bone hydrogel element 82d, and the brain hydrogel element 82e may be partially composed of additional component(s) that are different from the first component and/or the second component.

In one embodiment, the user may construct the phantom head 100 to be conductively similar to a human head, such as the head of the patient. That is, the user may construct the phantom head 100 such that the volume resistivity of the skin hydrogel element 82c is similar in volume resistivity to the skin of the patient, the volume resistivity of the bone hydrogel element 82d is similar in volume resistivity to the skull bone of the patient, and the brain hydrogel element 82e is similar in volume resistivity to the brain of the patient. In one embodiment, the user may also construct the phantom head 100 to include a target hydrogel element 82f having a volume resistivity similar in resistivity to the target, such as the target tumor.

In one embodiment, the user may construct the phantom head 100 to include one or more additional hydrogel element 82a-n modeling volume resistivity of other components in or around the patient's head, such as cartilage, eyes, hair, mucus, saliva, nerves, and the like. In one embodiment, the user may construct one or more hydrogel element 82a-n to simulate a portion of an organ, for example, the user may construct a first brain hydrogel element similar in volume resistivity to grey matter of the brain and a second brain hydrogel element similar in volume resistivity to white matter of the brain, or the user may construct a first bone hydrogel element similar in volume resistivity to bone marrow, a second bone hydrogel element similar in volume resistivity to spongy bone, and a third bone hydrogel element similar in volume resistivity to compact bone.

In one embodiment, the phantom head 100 may have one or more sensor 102a-n having a sensor lead 104a-n and associated with a particular location on or within the phantom head 100, such as a first sensor 102a having sensor lead 104a and associated with the target hydrogel element 82f and a second sensor 102b having sensor lead 104b and associated with the skin hydrogel element 82c. Additionally, each field-generating pad 70, such as the first field-generating pad 70a and the second field-generating pad 70b may include one or more sensor 102a-n. Each sensor 102a-n may include one or more of an electric field sensor, a voltage sensor, an ampere sensor, a temperature sensor, an electromagnetic field sensor, and/or the like. In one embodiment, by monitoring each sensor 102a-n, the user can determine an optimal placement of each of the one or more field-generating pad 70. The optimal placement of each of the one or more field-generating pad 70 may be determined by receiving data from the sensors 102a-n indicative of a maximized therapeutic benefit of the TTFields generated when one or more TTF signal is supplied to the first field-generating pad 70a, the second field-generating pad 70b, and any other field-generating pad 70 to be applied to the hydrogel phantom 78.

In some embodiments, the one or more sensor 102a-n may be placed in a plurality of different locations throughout the phantom hydrogel 78. For example, the sensor 102c is placed in the frontal region of the brain hydrogel element 82c. By placing one or more sensor 102a-n through the hydrogel phantom 78, the user may determine properties of the alternating electric field, e.g., TTField, at multiple locations within the phantom hydrogel 78. In other embodiments, at least one of the one or more sensor 102a-n may be placed at an intersection between two or more hydrogel elements 82a-n. By placing a sensor 102 at the intersection between two or more hydrogel elements 82a-n, the user may determine one or more property of the alternating electric field as it passes from a first hydrogel element 82 to a second hydrogel element 82.

In some embodiments, each of the one or more sensor 102a-n includes a sensor lead 104a-n communicably coupled to an external device 120. By accessing the external device 120, the user may be able to determine a value for one or more property of each sensor 102a-n. In other embodiments, however, each of the one or more sensor 102a-n does not include the sensor lead 104a-n, and may include a wireless transceiver communicably coupled to the external device 120 using a wireless communication topology conforming to the requirements of Bluetooth, RFID, WIFI, Xbee, Z-wave, and the like or some combination thereof, or any other wireless communication topology. In some embodiments, sensor 102 includes a sensor coupled to a processor through an analog to digital converter so as to provide digital signals that can be read and interpreted by the processor. In these embodiments, as sensor lead may couple the processor to the wireless transceiver to permit the processor to forward data and instructions to the external device 120 via the wireless transceiver.

In one embodiment, the phantom head 100 may have one or more simulated vein 108a-n. While the one or more simulated vein 108a-n is referenced to as a vein, the one or more simulated vein 108a-n may also simulate an artery, or another part of the human body designed to carry or convey the model liquid. In one embodiment, each of the one or more simulated vein 108a-n may include a tube, hose, or the like operable to circulate the model liquid, such as blood, or synthetic blood having electrical conductivity and/or volume resistivity properties similar to human blood, within the phantom head 100. In one embodiment, the synthetic blood also has thermal conductivity properties similar to human blood. In one embodiment, the model liquid is circulated while receiving data from the one or more sensor 102a-n.

In one embodiment, the hydrogel phantom 78 may be constructed to include one or more non-gel element 112, such as a medical device or the one or more simulated vein 108a-n for example. For example, if the hydrogel phantom 78 is the phantom head 100, the user may construct the phantom head 100 to include one or more non-gel element 112 that may be implanted in or placed on a patient's head, such as, for example, a medical device including a bone-anchored hearing aid, a cochlear implant, a metal plate such as one used to close a cranial defect, and/or the like. By constructing the hydrogel phantom 78 to include the one or more non-gel element 112, the user can measure changes in an electric field within the hydrogel phantom 78, such as the TTField, due to the one or more non-gel element 112.

In some embodiments, the one or more non-gel element 112 as a medical device, such as a pace maker, that actively generates an electric field. In one embodiment, by constructing the hydrogel phantom 78 as a chest cavity having one or more hydrogel element 82a-n with a volume resistivity similar to various organs within the chest cavity, and including the pace maker within the hydrogel phantom 78, the user can measure the electric field due to the field-generating pads 70a-n as well as any fluctuations in the electric field caused by electric signals generated by the pace maker.

In one embodiment, one or more additional field-generating pad 70 (not shown) may be attached to the phantom head 100. The generator 54, connected to each field-generating pad 70, may supply a first electrical signal having a first power and a first frequency to a first group of one or more field-generating pad 70, such as the first field-generating pad 70a and the second field-generating pad 70b, and supply a second electrical signal having a second power and a second frequency to a second group of one or more field-generating pads 70 attached to the phantom head 100 at the same instance in time. That is, the generator 54, may simultaneously supply the first electric signal to the first group and the second electric signal to the second group. While the above embodiments describe only the first group and the second group, it is understood that there may be more than two groups.

FIG. 3B is a cross-sectional diagram of another exemplary embodiment of the hydrogel phantom 78 of FIG. 2 depicted as a fluid container 130 configured to appropriately model electrical conductivity of an exterior of a biological component and an interior of a biological component. The fluid container 130 may be any shape including, but limited to, cube, rectangular prism, sphere, cone, cylindrical, or any fanciful shape. The fluid container 130 shown in FIG. 3B is a concave nearly hemispherical vessel formed to hold fluid.

The fluid container 130 includes at least one exterior wall 134 having an exterior surface 136 and an interior surface 138. The at least one exterior wall 134 may be formed from at least one hydrogel element 82. In some embodiments, the fluid container 130 includes a single wall formed of a first hydrogel element 82g. The hydrogel element 82g may be configured to approximate and/or model electrical conductivity of an exterior component of a biological component (e.g., skull, exterior skin of a torso and combinations thereof). For example, the hydrogel element 82g may be configured to approximate and/or model electrical conductivity of a skull of a human body, or non-human body. To that end, the hydrogel element 82g may be configured to approximate and/or model electrical conductivity of bone, skin, and/or brain matter.

The interior of the fluid container 130 may be filled or partially filled with a fluid solution 140. The fluid solution 140 may be configured to approximate and/or model electrical conductivity of an interior of a biological component (e.g., brain matter, blood, organs). For example, the fluid solution 140 may be configured to approximate and/or model electrical conductivity of brain matter (i.e., white matter and/or gray matter). In some embodiments, the fluid solution 140 may be configured to approximate an average conductivity of white matter and gray matter, for example. In some embodiments, the fluid solution 140 may be a saline solution, with salt content of the saline solution configured to approximate electrical conductivity of the interior of the biological component.

Referring to FIG. 3B, in some embodiments, one or more target hydrogel elements 82f may be formed on or attached to at least a portion of at least one movable probe 142. The target hydrogel element 82f may be configured to have a resistivity approximating one or more target tumor. The target hydrogel element 82f may be positioned at any point on the movable probe 142. The at least one movable probe 142 having the target hydrogel element 82f formed thereon or attached thereto may be positioned about the interior of the fluid container 130 and movable within the fluid solution 140 of the fluid container 130. For example, the at least one movable probe 142 may be positioned at a first site within the interior of the fluid container 130 wherein one or more measurements may be obtained relative to the target hydrogel element 82f. The at least one movable probe 142 may then be positioned at a second site within the interior of the fluid container 130 wherein one or more measurements may be obtained relative to the target hydrogel element 82f. Additionally, one or more target hydrogel elements 82f may be used with the at least one movable probe 142. To that end, the at least one movable probe 142 may be positioned at the first site wherein one or more measurements may be obtained relative to a first target hydrogel element 82f attached to the probe 142. The at least one movable probe 142 may be positioned at the second site wherein one or more measurements may be obtained relative to a second target hydrogel element 82f attached to the probe 142. It should be noted that additional probes, moveable or stationary, may be positioned within the interior of the fluid container 130 in accordance with the present disclosure.

In some embodiments, the fluid container 130, similar to the phantom head 100, may include one or more sensor 102a-n (shown in FIG. 3A), one or more simulated vein 108a-n (shown in FIG. 3A), and/or one or more non-gel element 112 (shown in FIG. 3A) positioned with the interior of the fluid container 130 (e.g., within the fluid solution 140 of the fluid container 130).

At least one movable probe 142 may be configured to be positioned within the fluid container 130 to measure the electric field within the interior of the fluid container 130. The interior of the fluid container 130 is bounded by the interior surface 138 of the exterior wall 134. Referring to FIGS. 2 and 3B, the one or more field-generating pad 70 may be attached to the exterior surface 136 of the exterior wall 134 of the fluid container 130. The generator 54, connected to each field-generating pad 70, may supply a first electrical signal having a first power and a first frequency to a first group of one or more field-generating pad 70, such as the first field-generating pad 70a and the second field-generating pad 70b, and supply a second electrical signal having a second power and a second frequency to a second group of one or more field generating pads 70 attached to the exterior of the fluid container 130. The at least one movable probe 142, having at least one target hydrogel element 82f thereon or attached thereto, may be configured to measure the electric field within the interior fluid container 130, and in some embodiments, within the target hydrogel element 82f during generation of the electrical signal from the generator 54.

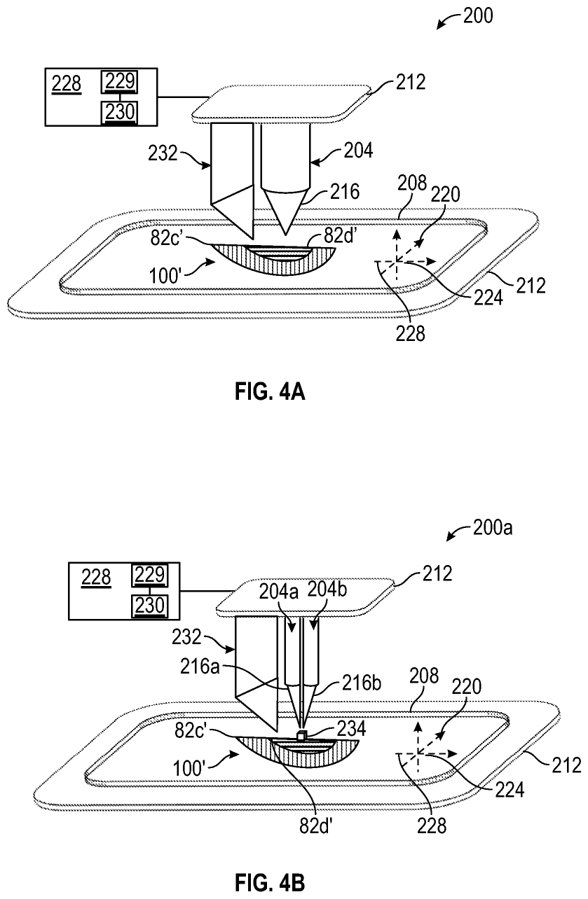

Referring now to FIG. 4A, shown therein is a diagram of an exemplary embodiment of a gel application system 200 constructed in accordance with the present disclosure. The gel application system 200 generally comprises one or more applicator 204 and a platform 208 moveably attached to a housing 212. Only one applicator 204 is shown for purposes of brevity; however, more than one applicator 204 may be utilized. The one or more applicator 204 further comprises at least a nozzle 216 to eject a conductive gel, described in more detail above, at an ejection rate. The platform 208 supports the hydrogel phantom 78 while the hydrogel phantom 78 is being constructed, depicted as partial phantom head 100′ having a partial skin hydrogel element 82c′ and a partial bone hydrogel element 82d′. In one embodiment, the first component and the second component (in liquid form) may be mixed within the applicator 204 and ejected as a liquid conductive gel from the nozzle 216 of the applicator 204.

In one embodiment, the applicator 204 may move in one of a first direction 220, a second direction 224, or a third direction 226, and combinations thereof. In one embodiment, the platform 208 may move in the first direction 220, the second direction 224, the third direction 226, or combination(s) thereof. The first direction 220 can be a y-direction, the second direction 224 can be an x-direction and the third direction 226 can be a z-direction. In one embodiment, the gel application system 200 includes a controller 228 to control movement of the platform 208 and/or to control movement of the applicator 204.

In some embodiments, the controller 228 is loaded with a three-dimensional model of a proposed hydrogel phantom having at least one proposed hydrogel element. In these embodiments, the three-dimensional model is provided with a plurality of voxels, with each voxel being a portion of one of the at least one proposed hydrogel element. Each of the voxels is provided with property information identifying (or used to determine) a particular resistance, impedance or conductance for the voxel. The property information is read by the controller 228 and can be used to create a voxel having the resistance, impedance or conductance.

In one embodiment, the controller 228 can be provided with circuitry, e.g., a memory 229, such as a non-transitory computer readable medium, communicably coupled to at least one processor 230. The memory 229 storing the three-dimensional model, and computer executable code configured to read the three-dimensional model, may be accessed by the processor 230. The processor 230, executing the computer executable code configured to read the three-dimensional model, may cause the applicator 204, of the platform 208, to move in one or more of the first direction 220, the second direction 224, or the third direction 228 and to cause the applicator 204 to eject the conductive gel at the ejection rate. In one embodiment, a computer system (not shown) is used to model the hydrogel phantom 78 as a plurality of voxels of the three-dimensional model and to communicate the three-dimensional model to the controller 228 where the three-dimensional model may then be stored in the memory 229. In one embodiment, the controller 228 is in communication with the one or more computer system to receive the three-dimensional model or a plurality of voxels forming the three-dimensional model.

In one embodiment, the gel application system 200 further includes a curing apparatus 232 to cause the first component and the second component (in liquid form) to cure, or polymerize, into a three-dimensional conductive gel of the one or more hydrogel element 82. The curing apparatus 232 may supply a curing agent, such as UV radiation, a laser, and/or an electron beam, for example, to a particular voxel 234 comprising the first component and the second component (in liquid form) where the particular voxel 234 is an uncured voxel of the three-dimensional model corresponding to a particular one of the one or more hydrogel element 82, shown in FIG. 4A as the partial skin hydrogel element 82c′ or the partial bone hydrogel element 82d′. The curing apparatus 232, by applying the curing agent, thereby causes the particular voxel 234 to polymerize into a three-dimensional conductive gel forming a portion of the particular one of the one or more hydrogel element 82.

In one embodiment, the user uses the controller 228 to cause the applicator 204 of the gel application system 200 to eject a first liquid hydrogel composed of the first component and the second component having the first ratio and the curing apparatus 232 to supply the curing agent to the first liquid hydrogel for a first duration and at a first intensity to form a first voxel of the partial skin hydrogel element 82c′ having a volume resistivity similar to the skin of the patient and to cause the applicator 204 of the gel application system 200 to eject a second liquid hydrogel composed of the first component and the second component having the second ratio and the curing apparatus 232 to supply the curing agent to the second liquid hydrogel for a second duration and at a second intensity to form a second voxel of the partial bone hydrogel element 82d′ having a volume resistivity similar to the skull bone of the patient.

In one embodiment, the user uses the controller 228 to cause the applicator 204 of the gel application system 200 to eject a liquid hydrogel composed of the first component and the second component having a particular ratio and the curing apparatus 232 supplies the curing agent to the liquid hydrogel for a particular duration and at a particular intensity on a voxel-by-voxel basis, that is, for a particular voxel, e.g., for each portion of one of the at least one hydrogel element 82, the applicator 204 ejects the liquid hydrogel and, after the liquid hydrogel for a particular voxel has been ejected, the curing apparatus 232 supplies the curing agent to the ejected liquid hydrogel. In one embodiment, the volume of each voxel may be determined based on one or more of a precision required in forming the hydrogel phantom 78, a volume of liquid hydrogel that can be cured in each voxel based, in part, on a viscosity of the liquid hydrogel, penetration limitations of the curing agent on the liquid hydrogel in the voxel, and the like. In one embodiment, each voxel of each hydrogel element 82 has a similar volume. In some embodiments, each voxel has a volume between 0.001 mm³ and 1 cm³. In some embodiments, each voxel has approximately the same volume whereas in other embodiments, not all voxels have the same volume. In some embodiments, each voxel has a width of between 0.01 mm and 1 cm.

In one embodiment, the controller 228, may slice the three-dimensional model into one or more layer where each layer is a plurality of substantially coplanar voxels and where each voxel of the plurality of substantially coplanar voxels corresponds to a volume of a particular hydrogel element 82a-n. For each voxel on a particular layer, the controller 228 may cause the applicator 204 to eject a liquid hydrogel, comprised of at least a first component and a second component, and to apply a curing agent to the voxel such that the voxel exhibits electrical conductivity, impedance, and/or volume resistivity similar to the voxel's corresponding volume of the particular hydrogel element 82a-n. In one embodiment, the controller 228 causes all or most of the plurality of substantially coplanar voxels to be ejected and cured on a layer-by-layer basis, that is, if the controller 228 slices the three-dimensional model into a first layer having a first plurality of substantially coplanar voxels and a second layer having a second plurality of substantially coplanar voxels, the controller 228 may cause most or all of the first plurality of substantially coplanar voxels at the first layer to be formed before the controller 228 causes most or all of the second plurality of substantially coplanar voxels at the second layer to be formed. In one embodiment, the computer system, may slice the three-dimensional model into one or more layer where each layer is a plurality of substantially coplanar voxels and where each voxel of the plurality of substantially coplanar voxels corresponds to a volume of a particular hydrogel element 82a-n.

In one embodiment, the nozzle 216 has an application distance determined by the distance between the nozzle 216 from the platform 208, and ejects conductive gel, such as hydrogel, (in liquid form) at an application pressure, and moves at an application velocity relative to the platform 208. By adjusting the application distance, the application pressure, and the application velocity, the voxel volume and/or voxel shape may be adjusted.

In one embodiment, more than one applicator 204 may be used to form more than one hydrogel phantom 78 simultaneously.

In one embodiment, one or more non-gel element, such as the one or more medical device, the one or more simulated vein 108a-n, and/or the like, may be placed on a particular layer of the partial phantom head 100′ as the partial phantom head 100′ is being constructed, or printed, by the gel application system 200. By attaching the one or more non-gel element during construction of the phantom head 100, the conductive gel may attach to the non-gel element when the conductive gel is cured, thereby preventing movement of the one or more non-gel element as movement of the non-gel element may introduce errors into the hydrogel phantom 78.

Referring now to FIG. 4B, shown therein is a diagram of an exemplary embodiment of a gel application system 200a constructed in accordance with the present disclosure. The gel application system 200a is similar in construction and function as the gel application system 200 described above and shown in FIG. 4A except that the applicator 204 includes a first applicator 204a and a second applicator 204b where the first applicator 204a is operable to eject the first component at a first rate (in liquid form) through a first nozzle 216a and the second applicator 204b is operable to eject the second component at a second rate (in liquid form) through a second nozzle 216b to a same location to cause the first and second component to mix and form a portion of one of the hydrogel element(s) 82. In this embodiment, by adjusting the first ejection rate of the first component and the second ejection rate of the second component for a period of time, the user can select a ratio between the first component and the second component to solidify into a solid, gel form. By repeating the steps of applying the first and second component, followed by curing the applied first and second components, the gel application system 200 can create the hydrogel elements 82a-n of the hydrogel phantom 78.

The gel application system 200a further comprises a platform 208 moveably attached to a housing 212. The platform 208 supports the hydrogel phantom 78 while the hydrogel phantom 78 is being constructed. The hydrogel phantom 78 is depicted as a partial phantom head 100′ having a partial skin hydrogel element 82c′ and a partial bone hydrogel element 82d′.

In some embodiments, the controller 228 is loaded with a three-dimensional model of a proposed hydrogel phantom having at least one proposed hydrogel element. In these embodiments, the three-dimensional model is provided with a plurality of voxels, with each voxel being a portion of one of the at least one proposed hydrogel element. Each of the voxels is provided with property information identifying (or used to determine) a particular resistance, impedance or conductance for the voxel. The property information is read by the controller 228 and can be used to create a voxel having the resistance, impedance or conductance. The controller 228 can be provided with a memory 229, such as a non-transitory computer readable medium, communicably coupled to at least one processor 230. The memory 229 storing the three-dimensional model, and computer executable code configured to read the three-dimensional model, may be accessed by the processor 230. The processor 230, executing the computer executable code configured to read the three-dimensional model, may cause the first applicator 204a and the second applicator 204b, of the platform 208, to move in one or more of the first direction 220, the second direction 224, or the third direction 228 and to cause the first applicator 204a to eject the first component (in liquid form) at the first rate through the first nozzle 216a and the second applicator 204b is operable to eject the second component (in liquid form) at the second rate through the second nozzle 216b.

In one embodiment, a computer system (not shown) is used to model the hydrogel phantom 78 as a plurality of voxels of the three-dimensional model and to communicate the three-dimensional model to the controller 228 where the three-dimensional model may then be stored in the memory 229. In one embodiment, the controller 228 is in communication with the one or more computer system to receive the three-dimensional model or a plurality of voxels forming the three-dimensional model. In one embodiment, the computer system may slice the three-dimensional model into one or more layer where each layer is a plurality of substantially coplanar voxels and where each voxel of the plurality of substantially coplanar voxels corresponds to a volume of a particular hydrogel element 82a-n.

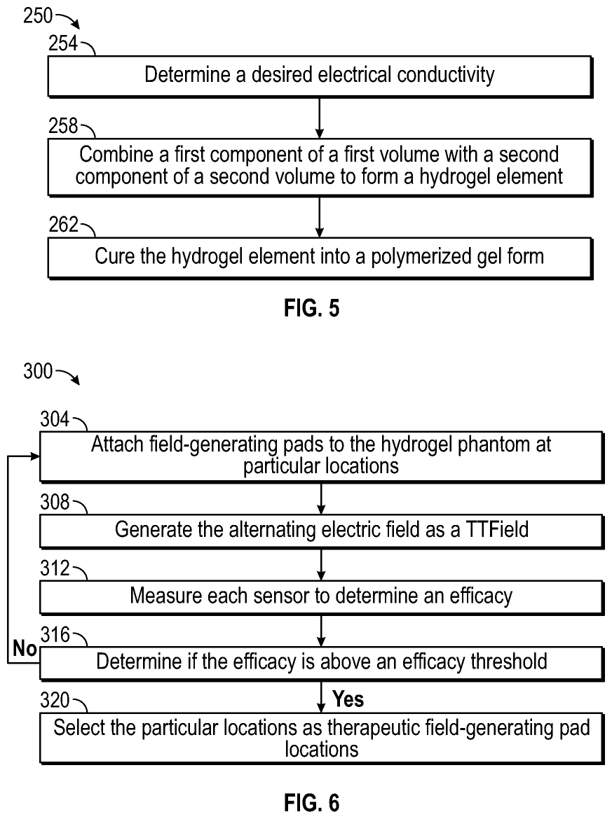

Referring now to FIG. 5, shown therein is an exemplary embodiment of a hydrogel phantom creation process 250 generally comprising the steps of: determining a desired volume resistivity (step 254), combining a first component hydrogel of a first volume and a second hydrogel of a second volume into a hydrogel element (step 258), and curing the hydrogel element to form a hydrogel phantom (step 262).

In one embodiment, determining a desired volume resistivity (step 254) may be performed by measuring a volume resistivity of a target region, such as a target tumor, of the patient. In one embodiment, determining a desired volume resistivity (step 254) may include selecting a volume resistivity for a particular portion of the patient, such as a particular organ, for a set of predetermined electrical conductivities for the particular organ. For example, if the particular organ is a liver, and it is pre-determined that, generally, a liver has a liver volume resistivity, then the pre-determined liver volume resistivity may be selected as the desired volume resistivity.

In one embodiment, combining the first component of the first volume and the second component of the second volume into the hydrogel element (step 258) generally comprises selecting a hydrogel component ratio, for example as determined to generate Table 1, where the particular ratio includes a volume resistivity approximate to or similar to the desired volume resistivity and selecting the first volume and the second volume such that the ratio of the first volume to the second volume is approximately equal to the hydrogel component ratio.

In one embodiment, combining the first component of the first volume and the second component of the second volume into the hydrogel element (step 258) may further include forming one or more voxel having the first volume of the first component and the second volume of the second component where each voxel is a discrete volume or portion of the hydrogel element.

In one embodiment, curing the hydrogel element to form a hydrogel phantom (step 262) generally comprises applying a curing agent to the hydrogel element for a particular duration. For example, the particular duration may be determined based in part on a curing duration corresponding to the hydrogel component ratio for the desired volume resistivity. In one embodiment, curing the hydrogel element to form a hydrogel phantom (step 262) may include curing each of the one or more voxel as each voxel is formed such that cured and solidified voxels collectively form the hydrogel element.

Referring now to FIG. 6, shown therein is an exemplary embodiment of a field-generating pad location placement process 300 generally comprising the steps of: attaching two or more field-generating pads to the hydrogel phantom 78 at particular locations (step 304), generating an alternating electric field having a frequency in a range of from about 50 kHz to about 1 MHz for a period of time (step 308), measuring one or more sensor to determine efficacy (step 312), determining if the efficacy is above an efficacy threshold (step 316), and if the efficacy is above the efficacy threshold, selecting the particular location of each of the two or more field-generating pads on the hydrogel phantom as a therapeutic field-generating pad location (step 320) otherwise, returning to step 304.

In one embodiment, attaching two or more field-generating pads to the hydrogel phantom (step 304) may be performed by the user and may include attaching the two or more field-generating pads at particular locations on the hydrogel phantom 78 and attaching one or more sensor to the hydrogel phantom 78. In one embodiment, generating an alternating electric field having a frequency in a range of from about 50 kHz to about 500 kHz for a period of time (step 308) includes accessing, by the user, the generator 54, or the control box 86, and causing the generator 54 to generate the alternating electric field.

In some embodiments, measuring one or more sensor to determine efficacy (step 312), may include measuring an electric field strength, or intensity, measuring a voltage, measuring an amperage, or measuring a temperature, or some combination thereof, for each of the one or more sensor 102a-n attached to the hydrogel phantom 78 to determine an efficacy of an applied alternating electric field at a target region. In one embodiment, the applied alternating electric field is the TTField and the target region is the target region of the field target 74. In some embodiments, measuring to determine efficacy may include obtaining one or more measurements of the electric field from the movable probe 142.

In one embodiment, determining if the efficacy is above an efficacy threshold (step 316) includes comparing the efficacy as determined in step 312 to an efficacy threshold and, if the efficacy is above the efficacy threshold, then selecting the current location of each of the two or more field-generating pads 70 on the hydrogel phantom 78 as a therapeutic field-generating pad location. If the efficacy is below the efficacy threshold, returning to attaching two or more field-generating pads to the hydrogel phantom 78 (step 304) and attaching the two or more field-generating pads to the hydrogel phantom in locations different from at least one of the particular locations. For example, if the efficacy threshold is a temperature threshold, then determining if the efficacy is below an efficacy threshold (step 316) includes comparing the efficacy, as determined by measuring the temperature, with the temperature threshold, and if the temperature exceeds the temperature threshold, return to step 304, otherwise continue to select the particular location of each of the two or more field-generating pads on the hydrogel phantom as a therapeutic field-generating pad location (step 320).

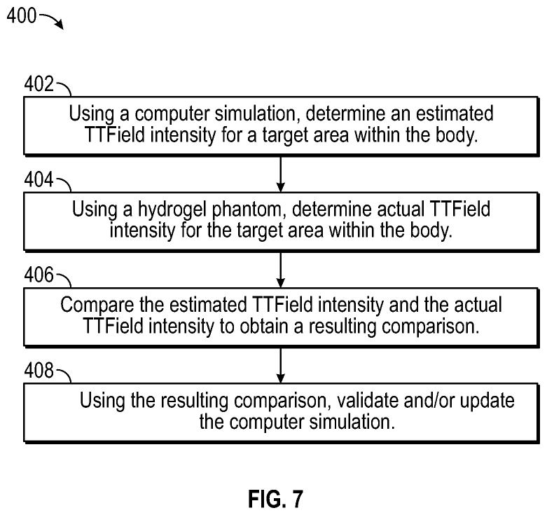

FIG. 7 is a flow chart of an exemplary method 400 for validating a simulation of TTField intensity (i.e., estimated TTField intensity predicted within a computer model), in accordance with the present disclosure. The following may also be validated in lieu of or in addition to the TTField intensity: voltage, amperage, temperature, or some combination thereof in accordance with the present disclosure.

In a step 402, one or more computer simulation may determine an estimated TTField intensity for a target area within the body. Generally, the computer simulation determines position(s) for electrodes for TTField treatment. One or more computer models of electrical conductivity within the body (e.g., head, torso) may be generated by obtaining CT scan and/or MRI images of a particular portion of the body. For example, disclosures within the following patents and patent publications detail segmentation of CT scans and/or MRI images to provide computer models of conductivity used to determine positions for electrodes in TTFields treatment: U.S. Pat. No. 10,188,851, filed on Oct. 27, 2016; U.S. Patent Publication No. 2020/0146586, filed on Nov. 12, 2019; and, U.S. Patent Publication No. 2020/0023179, filed on Jul. 18, 2019, which are all hereby incorporated by reference in their entirety. Within the computer simulation, an estimated TTField intensity may be determined for a target area (e.g., tumor) within the body based on simulated positioning of the electrodes.

In a step 404, using the hydrogel phantom 78, actual TTField intensity within the hydrogel phantom 78 may be obtained in accordance with the present disclosure. Field generating pads 70a and 70b may be positioned about the hydrogel phantom 78 based on positioning of the electrodes with the computer simulation or model. An alternating electric field may be applied to the hydrogel phantom 78 with the field generating pads 70a and 70b. Actual TTField intensity may be measured related to the alternating electric field passing through at least a portion of the hydrogel phantom 78.

In a step 406, the estimated TTField intensity of the computer simulation and the actual TTField intensity of the hydrogel phantom 78 may be compared to provide a resulting comparison output. In a step 408, the resulting comparison output may validate the computer simulation of the estimated TTField intensity and/or the resulting comparison output may be used to update the computer simulation. For example, the resulting comparison output may validate the estimated TTField intensity provided by the computer simulation if the difference between the actual TTField intensity and the estimated TTField intensity is within a pre-determined threshold. In some embodiments, the resulting comparison may be used to adjust or calibrate the computer simulation (e.g., update one or more algorithms within the computer simulation).

From the above description, it is clear that the inventive concepts disclosed and claimed herein are well adapted to carry out the objects and to attain the advantages mentioned herein, as well as those inherent in the invention. While exemplary embodiments of the inventive concepts have been described for purposes of this disclosure, it will be understood that numerous changes may be made which will readily suggest themselves to those skilled in the art and which are accomplished within the spirit of the inventive concepts disclosed and claimed herein.

What is claimed is:

1. A hydrogel phantom, comprising:
a plurality of connected hydrogel elements, a first one of the hydrogel elements having a first electrical impedance and a second one of the hydrogel elements having a second impedance with the first impedance different from the second impedance.

2. The hydrogel phantom of claim 1, wherein the plurality of connected hydrogel elements are in the form of a patient's body part.

3. The hydrogel phantom of claim 1, wherein at least one of the hydrogel elements is in the form of a tumor, and the at least one of the plurality of adjacently disposed hydrogel elements has an impedance mimicking the impedance of the tumor.

4. The hydrogel phantom of claim 1, wherein the plurality of connected hydrogel elements are in the shape of a human head.

5. The hydrogel phantom of claim 1, wherein each of the plurality of connected hydrogel elements include a predetermined ratio of a first component and a second component.

6. The hydrogel phantom of claim 1, further comprising a non-gel element communicating with at least one of the plurality of connected hydrogel elements.

7. The hydrogel phantom of claim 6, wherein the non-gel element is a medical device communicating with at least one of the plurality of adjacently disposed hydrogel elements.

8. The hydrogel phantom of claim 6, wherein the non-gel element is implanted within the plurality of adjacently disposed hydrogel elements.

9. A method, comprising:
receiving a 3-dimensional model of an object, the 3-dimensional model having a plurality of voxels, with each voxel provided with property information identifying or being usable to determine at least one of an impedance or a resistance for the voxel; and
operating a gel application system to create a hydrogel phantom with the 3-dimensional model, by creating hydrogel elements within the hydrogel phantom corresponding to voxels within the 3-dimensional model.

10. A method, comprising:
attaching field-generating pads to a hydrogel phantom at particular locations on the hydrogel phantom, the hydrogel phantom having a plurality of connected hydrogel elements, a first one of the hydrogel elements having a first electrical impedance and a second one of the hydrogel elements having a second impedance with the first impedance different from the second impedance;
applying an alternating electric field to the hydrogel phantom with the field generating pads;
measuring of at least one property related to the alternating electric field passing through at least a portion of the hydrogel phantom with a plurality of sensors; and
performing at least one of the following steps:
determining an efficacy of the alternating electric field on a target region within the hydrogel phantom; and
modeling the alternating electric field passing through at least a portion of the hydrogel phantom using data measured by the plurality of sensors.

11. The method of claim 10, wherein applying an alternating electric field includes applying a tumor treating field to the hydrogel phantom with the field generating pads.

12. The method of claim 10 further comprising calculating a specific absorption rate of the alternating electric field by the hydrogel phantom based at least in part on the measured at least one property related to the alternating electric field.

13. The method of claim 10 further comprising attaching the plurality of sensors on or within the hydrogel phantom and associated with a particular portion of the hydrogel phantom, each sensor providing at least one property.

14. The method of claim 13 wherein measuring of at least one property related to the alternating electric field further includes measuring at least one of the plurality of sensors to determine the at least one property.

15. The method of claim 14 wherein measuring of at least one property related to the alternating electric field further includes measuring at least one of the plurality of sensors to determine a temperature related to the alternating electric field passing through the particular portion of the hydrogel phantom.

16. The method of claim 14 wherein measuring of at least one property related to the alternating electric field further includes measuring at least one of the one or more sensor to determine an electrical property related to the alternating electric field passing through the particular portion of the hydrogel phantom.

17. The method of claim 14 wherein measuring of at least one property related to the alternating electric field further includes measuring at least one of the one or more sensor to determine a magnetic property related to the alternating electric field passing through the particular portion of the hydrogel phantom.

18. The method of claim 14, wherein applying an alternating electric field includes applying a tumor treating field to the hydrogel phantom with the field generating pads.

19. The method of claim 18, wherein modeling the tumor treating field includes determining an efficacy of the alternating electric field on a target region within the hydrogel phantom.

20. A method, comprising:
attaching field-generating pads to a hydrogel phantom at pre-determined locations based on a computer simulation, the hydrogel phantom having a plurality of hydrogel elements, a first one of the hydrogel elements having a first electrical impedance and a second one of the hydrogel elements having a second impedance with the first impedance different from the second impedance;
applying an alternating electric field to the hydrogel phantom with the field generating pads;
measuring TTField intensity related to the alternating electric field passing through at least a portion of the hydrogel phantom to obtain an actual TTField intensity; and,
comparing the actual TTField intensity to an estimated TTField intensity obtained from the computer simulation.
